(19) United States
(12) Patent Application Publication
(10) Pub. No.: US 2020/0275845 A1
(43) Pub. Date: Sep. 3, 2020

(54) BLOOD-PRESSURE MEASUREMENT APPARATUS AND BLOOD-PRESSURE MEASUREMENT METHOD

(71) Applicants: KABUSHIKI KAISHA TOSHIBA, Tokyo (JP); Toshiba Electronic Devices & Storage Corporation, Tokyo (JP)

(72) Inventor: Ken Kawakami, Kawasaki Kanagawa (JP)

(21) Appl. No.: 16/804,134

(22) Filed: Feb. 28, 2020

(30) Foreign Application Priority Data
Feb. 28, 2019 (JP) 2019-035280
May 17, 2019 (JP) 2019-093999
Feb. 19, 2020 (JP) 2020-026521

(51) Int. Cl. A61B 5/026; A61B 5/022; A61B 5/021

(52) U.S. Cl. CPC A61B 5/0261; A61B 5/022; A61B 5/02416; A61B 5/02141; A61B 5/02125

(57) ABSTRACT

A blood-pressure measurement apparatus according to an embodiment comprises a measurer and a blood-pressure acquirer. The measurer is configured to measure a pulse of a subject based on a received-light signal scattered in a body of the subject when a light signal in a predetermined frequency band is irradiated. The blood-pressure acquirer is configured to acquire a diastolic blood pressure based on a first value and a second value, the first value corresponding to a blood flow of the subject in a first time period in a time period from a first reference time at which a value obtained by first-order differentiation of the pulse with respect to a time becomes the maximum to a second reference time at which a next pulse rises, the second value corresponding to a vascular resistance of the subject.

### CROSS-REFERENCE TO RELATED APPLICATIONS

This application is based upon and claims the benefit of priority from Japanese Patent Application No. 2019-035280, filed on Feb. 28, 2019, Japanese Patent Application No. 2019-093999, filed on May 17, 2019 and Japanese Patent Application No. 2020-26521, filed on Feb. 19, 2020; the entire contents of which are incorporated herein by reference.

### FIELD

Embodiments of the present invention relate to a blood-pressure measurement apparatus and a blood-pressure measurement method.

### BACKGROUND

There is known a photoplethysmogram (PPG) sensor that detects a pulse associated with a heartbeat by measuring a change of a blood volume in an artery and capillaries which corresponds to a change of a heart rate. A method that detects the heart rate by using the PPG sensor based on the blood volume passing through a tissue at each pulse beat is called “blood volume pulse (BVP) measurement”.

There is generally known a method that estimates a blood pressure based on feature points of a waveform shape of a blood volume pulse. However, the waveform of the blood volume pulse fluctuates depending on a state of activity or a mental state of a subject, so that disturbance occurs in the blood volume pulse. While the blood volume pulse is disturbed, it is impossible to accurately measure the feature points or the like, and the accuracy of measurement of the blood pressure is deteriorated.

### BRIEF DESCRIPTION OF THE DRAWINGS

FIG. 1 is a block diagram illustrating a schematic configuration of a blood-pressure measurement apparatus according to a first embodiment;

FIG. 2 is a diagram illustrating an example of a watch type blood-pressure measurement apparatus;

FIG. 3 is a diagram illustrating an example of a blood volume pulse measured by a measurer;

FIG. 4 is a diagram illustrating a blood vessel obtained by approximation by a cylindrical tube;

FIG. 5 is a diagram illustrating a relation between an example of a pulse waveform and a radius of a cylindrical tube model;

FIG. 6 is a diagram schematically illustrating a change of a radius of a cylindrical tube which is associated with a change of a blood vessel volume;

FIG. 7A is a diagram schematically illustrating a flow rate from a point P_{S} to a point P_{E};

FIG. 7B is a diagram schematically illustrating a flow rate from the point P_{E} to a point P_{H};

FIG. 8 is a diagram illustrating an example of information acquired by a feature-point processor;

FIG. 9A is a diagram illustrating measured data of a subject having a relatively high blood pressure;

FIG. 9B is a diagram illustrating measured data of a subject having a relatively low blood pressure;

FIG. 10 is a flowchart illustrating processing by the blood-pressure measurement apparatus;

FIG. 11 is a diagram illustrating a relation between an example of a pulse waveform and a radius of a cylindrical tube model according to a second embodiment;

FIG. 12 is a diagram schematically illustrating a change of a radius of a cylindrical tube which is associated with a change of a blood vessel volume from the point P_{S} to a point P_{L} via a point P_{D};

FIG. 13A is a diagram schematically illustrating a flow rate from the point P_{S} to the point P_{H} via the point P_{E};

FIG. 13B is a diagram schematically illustrating a flow rate from the point P_{H} to the point P_{D}; and

FIG. 14 is a diagram illustrating an example of information acquired from a pulse by a feature-point processor according to the second embodiment.

### DETAILED DESCRIPTION

A blood-pressure measurement apparatus according to an embodiment comprises a measurer and a blood-pressure acquirer. The measurer is configured to measure a pulse of a subject based on a received-light signal scattered in a body of the subject when a light signal in a predetermined frequency band is irradiated. The blood-pressure acquirer is configured to acquire a diastolic blood pressure based on a first value and a second value, the first value corresponding to a blood flow of the subject in a first time period in a time period from a first reference time at which a value obtained by first-order differentiation of the pulse with respect to a time becomes the maximum to a second reference time at which a next pulse rises, the second value corresponding to a vascular resistance of the subject.

### First Embodiment

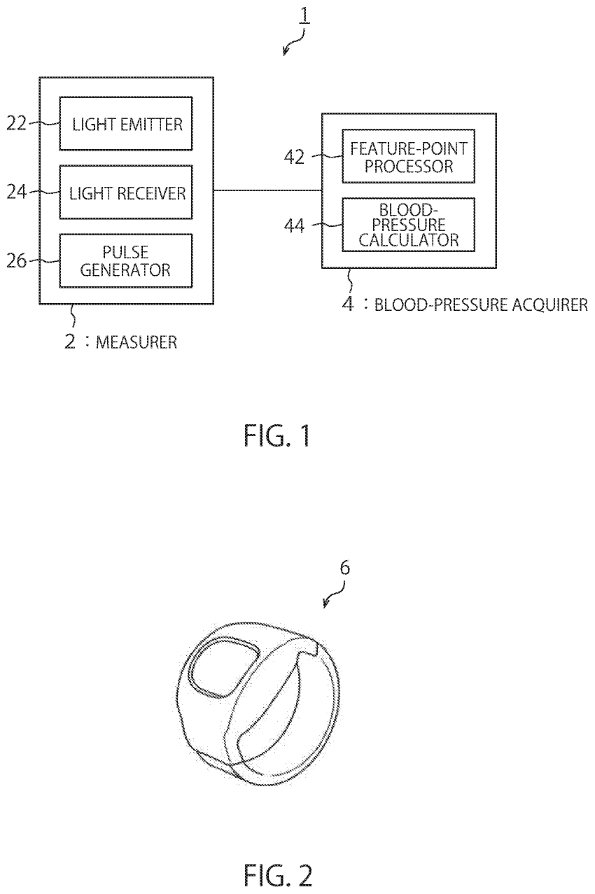

FIG. 1 is a block diagram illustrating a schematic configuration of a blood-pressure measurement apparatus 1 according to a first embodiment. The blood-pressure measurement apparatus 1 includes a measurer 2 and a blood-pressure acquirer 4. The blood-pressure measurement apparatus 1 can be incorporated in a watch type biological measurement apparatus 6 illustrated in FIG. 2, for example. The biological measurement apparatus 6 may be arranged on an upper arm, the chest, or the like.

The measurer 2 measures a change of a blood volume in an artery and capillaries which is associated with a change of a heart rate of a subject, to acquire information on a blood volume pulse associated with a heartbeat. The blood volume pulse may be simply referred to as “pulse” in the following descriptions. The measurer 2 includes a light emitter 22, a light receiver 24, and a pulse generator 26. The light emitter 22 includes an LED (Light Emitting Device) that emits a light signal in a specific wavelength band (a green band, a near infrared band, or the like), for example. The light receiver 24 receives a signal after the light signal from the light emitter 22 is absorbed or reflected/scattered in the body of the subject. The pulse generator 26 generates a pulse at each heartbeat based on a received-light signal.

When the amount of light emission of the light signal fluctuates, the received-light amount of the received-light signal also fluctuates. Therefore, the pulse generator 26 separates the received-light signal into a DC component and an AC component, and generates a pulse based on an AC/DC ratio. Accordingly, the generated pulse is dimensionless data.

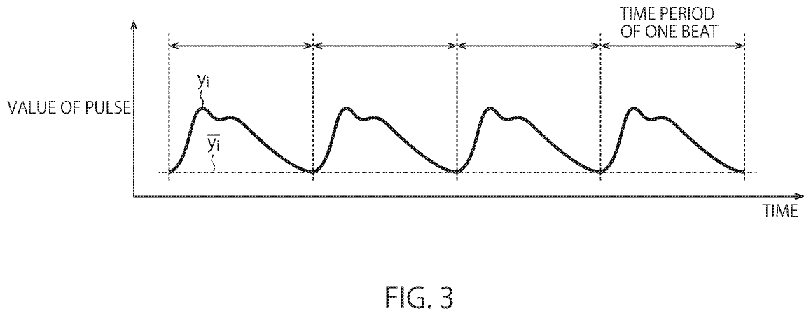

FIG. 3 is a diagram illustrating an example of a blood volume pulse measured by the measurer 2. The vertical axis represents a value of pulse and the horizontal axis represents a time. As illustrated in FIG. 3, the pulse repeats fluctuation every heartbeat. A pulse yᵢ at the i-th beat is formed by an AC component that fluctuates periodically and a DC component V.

The blood-pressure acquirer 4 acquires a blood pressure of a subject based on the pulse. This blood-pressure acquirer 4 includes a feature-point processor 42 and a blood-pressure calculator 44.

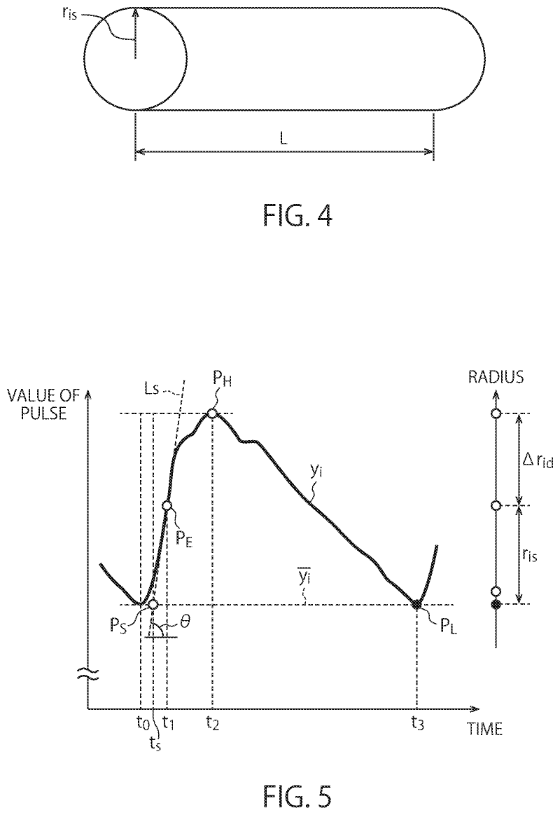

First, a model used in the blood-pressure acquirer 4 is described referring to FIGS. 4 to 7B. FIG. 4 is a diagram illustrating a blood vessel model. The blood vessel model illustrated in FIG. 4 is obtained by approximation by a cylindrical tube having a radius rᵢₛ and a length L. Fluctuation of a blood pressure is fluctuation of a pressure applied to a vascular wall by blood ejected from the heart. This blood-pressure fluctuation is linked with the pulse yᵢ.

A relation among a pressure difference ΔP, a flow rate Q, and a resistance R of the cylindrical tube is derived from the Navier-Stokes equations and is represented by Expression (1).

ΔP=QR (1)

The blood-pressure acquirer 4 calculates values corresponding to the flow rate Q and the resistance R by using the pulse yᵢ based on the cylindrical tube model to acquire the blood pressure of the subject. In general, a human blood pressure is evaluated by using a systolic blood pressure SBP that is the maximum pressure in a blood vessel in a systolic phase of the heart, a diastolic blood pressure DBP that is the minimum pressure in the blood vessel in a diastolic phase of the heart, and a pulse pressure PP obtained by subtracting the diastolic blood pressure from the systolic blood pressure.

FIG. 5 is a diagram illustrating a relation between an example of a pulse waveform and a radius of a cylindrical tube model according to the first embodiment. The left part of FIG. 5 illustrates an example of a normal pulse waveform for one beat. The vertical axis represents a value of pulse and the horizontal axis represents a time. The right part of FIG. 5 illustrates the radius of the cylindrical tube model. A change of a blood vessel volume is represented by the radius rᵢₛ and a change Δr_{id}. That is, the radius rᵢₛ is the radius of a blood vessel at a point P_{E}, and Δr_{id} is increase of the radius associated with increase of the volume from the point P_{E} to a point P_{H}.

In the normal pulse yᵢ, the amplitude starts at a bottom position (t₀), increases substantially monotonically and reaches a maximum peak (t₂), thereafter monotonically decreases and reaches a bottom position (t₃), and ends. Here, the suffix i is the number for identifying each pulse in blood volume pulse data. That is, the suffix i indicates data corresponding to a pulse at the i-th beat. Although calculation for each beat is performed in the calculation according to the present embodiment, a manner of calculation is not limited thereto. Data for several beats may be averaged and be subjected to calculation, for example.

t₁ is a time between to and t₂, at which a value obtained by the first-order differentiation of the pulse y₃ with respect to a time becomes the maximum. This t₁ corresponds to an equilibrium point of a displacement r(t) of an equation of viscoelastic motion represented by Equation (4) described later.

tₛ is a time based on a value obtained by dividing a first difference value obtained by subtracting a direct-current component

y̅ᵢ

from a value yᵢₛ of the pulse yᵢ at the time t₁ by the maximum first-order differentiation value

y′ᵢ

as represented by Equation (2). Here, the first-order differentiation value

y′ᵢ

is calculated by Equation (15) described later, for example.

tₛ=t₁−(yᵢₛ−y̅ᵢ)/y′ᵢ (2)

A line Ls is a tangent at the point P_{E}. That is, tan θ calculated by an angle θ between the line Ls and a line horizontal to the horizontal axis corresponds to this first-order differentiation value

y′ᵢ

P_{S}, P_{E}, P_{H}, and P_{L} denote points that correspond to the times tₛ, t₁, t₂, and t₃, respectively. The time t_{L} according to the present embodiment corresponds to a first time, the time t₂ corresponds to a second time, and the time t₀ or tₛ corresponds to a third time.

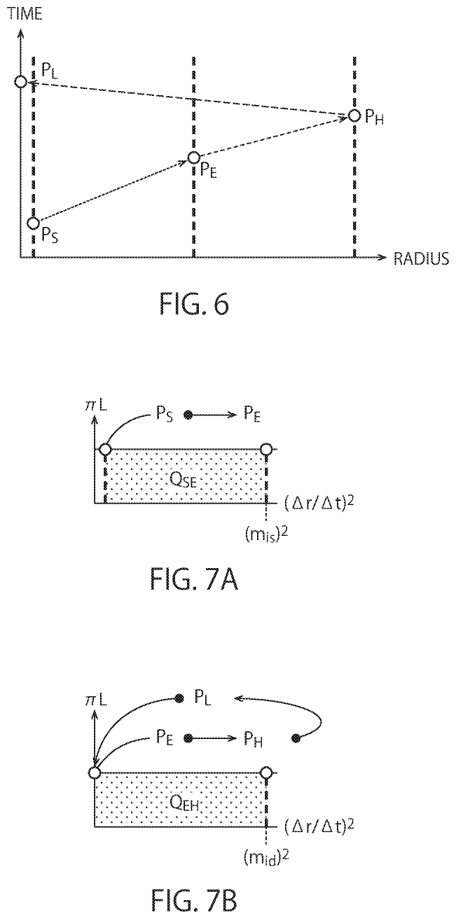

FIG. 6 is a diagram schematically illustrating a change of a radius of a cylindrical tube which is associated with a change of a blood vessel volume from the point P_{S} to the point P_{L}. The vertical axis represents a time and the horizontal axis represents a change of the radius from the point P_{S}. The radius increases from the point P_{S} to the point P_{H} with the time, and thereafter decreases.

A blood flow is measured as a volumetric flow rate Q based on the radius r in accordance with the volume change of a blood vessel model. Therefore, the flow rate Q can be defined by using an average rate of change of the radius per unit time (Δr/Δt). Δt represents a time change amount of t, and Δr represents a change amount of the radius r for Δt.

FIG. 7A is a diagram schematically illustrating a flow rate Q_{SE} from the point P_{S} to the point P_{E}. FIG. 7B is a diagram schematically illustrating a flow rate Q_{EH} from the point P_{E} to the point P_{H}. The horizontal axis represents the square of an average rate of change (Δr/Δt), and the vertical axis represents a value obtained by multiplying the length L and n. An average rate of change mᵢₛ is represented by Equation (8) described later. The average rate of change mᵢₛ in FIG. 7A is a value obtained by dividing the radius rᵢₛ from the point P_{S} to the point P_{E} by a value obtained by subtracting the time t, from the time t₁. An average rate of change m_{id} in FIG. 7B is a value obtained by dividing Δr_{id} by a value obtained by subtracting the time t_{b} from the time t₂. Further, a flow rate Q_{EL} (not illustrated) from the point P_{H} to the point P_{L} can be obtained by Equation (3) by using a time T_{id2} from the point P_{H} to the point P_{L}, the resistance R, compliance C of a blood vessel, and the flow rate Q_{EH}. In Equation (3), the term of the Napier's constant is known as a method of representing a pressure drop after a systolic phase in a two-element Windkessel model.

Q_{EL}=Q_{EH}e^{-T}^{_{id2}}^{/RC}

A displacement r(t) of a vascular wall, that is, a displacement of the radius r(t) is linked with a value of the pulse yᵢ. Further, a pressure can be approximated by the displacement r(t) of the vascular wall, and the displacement of the radius r(t) is equivalent to the equation of viscoelastic motion represented by Equation (4). That is, in a case of approximating a blood vessel by a cylindrical tube, a blood pressure can be calculated based on information on the pulse yᵢ.

$$\frac{{d}^{2} r ( t )}{{dt}^{2}} = - b \frac{dr ( t )}{dt} - kr ( t ) - F ( t ) \qquad ( 4 )$$

Here, b is a viscosity constant, k is an elastic constant, and elasticity of a blood vessel is reflected on them. The left side in Equation (4) represents an entire force in the Newton's second law. The first term in the right side represents a damping force, the second term represents a restoring force, and the third term represents a force by the Windkessel effect. An equilibrium position of the displacement r(t) corresponds to the point P_{E}.

In a systolic phase of the heart, a vascular wall is displaced mainly by the Windkessel effect from rising of the pulse yᵢ to the point P_{E}. Meanwhile, from the point P_{E} to the point P_{H}, the vascular wall is displaced by the damping force and the restoring force. At the point P_{H}, the Windkessel effect and the damping force can be ignored.

Therefore, in the systolic phase of the heart, expansion of the radius rᵢₛ to the point P_{E} is caused mainly by the Windkessel effect. Accordingly, a force generated in the systolic phase of the heart, that is, a systolic blood pressure SBP is reflected on the flow rate Q_{SE}. Meanwhile, a diastolic blood pressure DBP is reflected on the flow rate Q_{EL}. Since the flow rate Q_{EL} is a lower limit of the force by the Windkessel effect, it is obtained from the flow rate Q_{EH} from the point P_{E} to the point P_{H}. The flow rate Q_{EH} according to the present embodiment corresponds to a first value, the resistance R corresponds to a second value, and the flow rate Q_{SE} corresponds to a third value.

Accordingly, in the present embodiment, a systolic blood pressure SBPᵢ at a heartbeat i is modeled by Equation (5). R represents a value that reflects a peripheral circulation resistance in observation of flow rates Q_{SEi} and Q_{ELi}. Here, a and a are constants.

SBPᵢ=aQ_{SE}_{ᵢ}Rᵢ+DBPᵢ+α (5)

Further, a diastolic blood pressure DBP; is modeled by Equation (6).

DBPᵢ+bQ_{EL}_{ᵢ}Rᵢ+β (6)

Here, b and β are constants. The constants a, α, b, and β can be calculated by the least squares method, for example, in such a manner that values of measurement by the blood-pressure measurement apparatus 1 and data measured by a medical instrument (for example, a wrist-cuff type) simultaneously with the measurement by the blood-pressure measurement apparatus 1 are coincident with each other. Once the constants a, α, b, and β are determined, calculation of the constants a, α, b, and β is not necessary in measurement performed later. A pulse pressure PP; is a value obtained by subtracting the diastolic blood pressure DBPᵢ from the systolic blood pressure SBPᵢ.

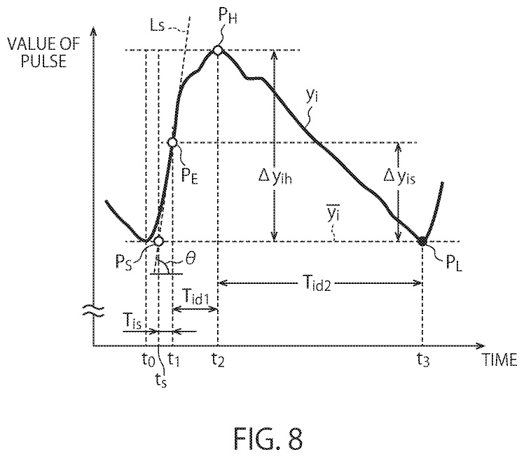

The model used by the blood-pressure acquirer 4 according to the present embodiment has been described above. The detailed configurations of the blood-pressure acquirer 4 are described below. FIG. 8 is a diagram illustrating an example of information acquired by the feature-point processor 42 from the pulse yᵢ. The vertical axis represents a value of pulse and the horizontal axis represents a time.

The feature-point processor 42 detects to as a rising time and t₂ as a time of the maximum peak. The feature-point processor 42 also calculates t₁ between to and t₂, at which a value obtained by the first-order differentiation of a pulse with respect to a time becomes the maximum.

Further, the feature-point processor 42 calculates a first difference value Δyᵢₛ obtained by subtracting the direct-current component

y̅ᵢ

from a value of the pulse yᵢ at the time t₁ and a second difference value Δyᵢₕ obtained by subtracting the direct-current component

y̅ᵢ

from a value of the pulse yᵢ at the time t₂.

The feature-point processor 42 calculates a time Tᵢₛ by using Equation (7). Ti, is a time obtained by dividing the first difference value Δyᵢₛ by tan θ corresponding to a differential value of the time t₁. The second rising time t; obtained by subtracting Tᵢₛ from t₁ is then calculated. T_{id1} is a time obtained by subtracting t from the time t₂, and T_{id2} is a time obtained by subtracting t₂ from the time t₃.

$${T}_{is} = \frac{Δ {y}_{is}}{tan θ} \qquad ( 7 )$$

The shape of the pulse yᵢ at the rising time t₀ is highly different between individuals, and changes gently for some people and changes steeply for other people. Therefore, a difference value between the rising time t₀ and the time t₀ can easily fluctuate because of the difference between individuals. Meanwhile, in the time difference Tᵢₛ between the second rising time tₛ and the time t₁, fluctuation because of the difference between individuals is reduced, so that the time difference Tᵢₛ has a stable value. Therefore, calculation of a flow rate uses this time difference Tᵢₛ. For a certain shape of the pulse yᵢ, the time Tᵢₛ may be calculated as a time difference between the time t₀ and the time t₁. Accordingly, calculation can be simplified.

The blood-pressure calculator 44 acquires a diastolic blood pressure DBP based on the flow rate Q_{EH} (the first value) corresponding to a blood flow of a subject from the first time t₁ at which a value obtained by the first-order differentiation of the pulse yᵢ with respect to a time becomes the maximum to the second time t₂ of the maximum peak of the pulse, and the resistance R (the second value) corresponding to a vascular resistance of the subject, as represented by Equations (3) and (6). That is, the blood-pressure calculator 44 calculates a value obtained by multiplying a product of the flow rate Q_{EL} based on the flow rate Q_{EH} and the resistance R by a predetermined constant b and further adding a predetermined constant 13, as the diastolic blood pressure DBP. The resistance R is calculated based on Equations (14) and (15) described later. Further, this blood-pressure calculator 44 acquires a systolic blood pressure SBP further based on the flow rate Q_{SE} (the third value) corresponding to a blood flow from the third time tₛ or to of rising of the pulse to the time t₁ by using Equation (5). The first time according to the present embodiment corresponds to a first reference time, the second time corresponds to a fourth reference time, and the third time corresponds to a third reference time.

In more detail, the blood-pressure calculator 44 calculates rᵢₛ based on Equation (8) and calculates the average rate of change mᵢₛ based on Equation (9). The blood-pressure calculator 44 calculates the flow rate Q_{SE} based on Equation (10). Here,

Δyᵢₛ/y̅ᵢ

is proportional to a blood vessel volume at the point P_{E}. In this manner, the blood-pressure calculator 44 acquires the systolic blood pressure SBP based on Equations (5) and (10). The first difference value Δyᵢₛ of the pulse yᵢ, the direct-current component

y̅ᵢ

and the time T, that are calculated at this time can be stably and simply calculated, also with respect to fluctuation of the pulse yᵢ. Therefore, it is possible to acquire the systolic blood pressure SBPᵢ simply and accurately. G is a constant.

$$\begin{matrix}{r}_{is} = \sqrt{\frac{G}{L}} \sqrt{\frac{Δ {y}_{is}}{π {\overline{y}}_{i}}} & ( 8 ) \\ {m}_{is} \sim \frac{{r}_{is}}{{T}_{is}} & ( 9 ) \\ {Q}_{SE} = π {L ( {m}_{is} )}^{2} = π {L ( \frac{{r}_{is}}{{T}_{is}} )}^{2} = π L \frac{\frac{G}{L} \frac{Δ {y}_{is}}{π {\overline{y}}_{i}}}{{( {T}_{is} )}^{2}} = \frac{G Δ {y}_{is}}{{{\overline{y}}_{i} ( {T}_{is} )}^{2}} & ( 10 )\end{matrix}$$

The blood-pressure calculator 44 calculates Δr_{id} based on Equation (11) and calculates the average rate of change m_{id} based on Equation (12). The blood-pressure calculator 44 further calculates the flow rate Q_{EL} from the point P_{H} to the point P_{L} based on Equation (2). In this manner, the blood-pressure calculator 44 acquires the diastolic blood pressure DBPᵢ based on Equations (6) and (13). The first difference value Δyᵢₛ, the second difference value Δyᵢₕ, the direct-current component

y̅ᵢ

and the times Tᵢₛ and T_{id} that are calculated at this time can be stably and simply calculated, also with respect to fluctuation of the pulse yᵢ. Therefore, it is possible to acquire the systolic blood pressure SBP_{E} simply and accurately.

$$\begin{matrix} Δ {r}_{id} = \sqrt{\frac{G}{4 L}} \frac{Δ {y}_{ih} - Δ {y}_{is}}{Δ {y}_{is}} \sqrt{\frac{Δ {y}_{is}}{π {\overline{y}}_{i}}} & ( 11 ) \\ {m}_{id} = \frac{Δ {r}_{id}}{{T}_{id 1}} & ( 12 ) \\ {Q}_{EH} = π {L ( {m}_{id} )}^{2} = π {L ( \frac{Δ {r}_{id}}{{T}_{id 1}} )}^{2} = π {L ( \frac{\sqrt{\frac{G}{4 L}} \frac{Δ {y}_{i h} - Δ {y}_{is}}{Δ {y}_{i s}} \sqrt{\frac{Δ {y}_{is}}{π {\overline{y}}_{i}}}}{{T}_{i d l}} )}^{2} = \frac{\frac{G}{4} {( \frac{Δ {y}_{ih} - Δ {y}_{is}}{Δ {y}_{is}} )}^{2} \frac{Δ {y}_{is}}{{\overline{y}}_{i}}}{{( {T}_{id 1} )}^{2}} & ( 13 )\end{matrix}$$

In this manner, the first value Q_{EL} and the third value Q_{SE} are values based on

Δyᵢₛ/y̅ᵢ

corresponding to the blood vessel volume of a subject at the time tₛ. The blood vessel volume is a value based on a value obtained by dividing the first difference value Δyᵢₛ by the direct-current component

y̅ᵢ

That is, the first value Q_{EL} is a value based on a product of a value obtained by subtracting the first difference value Δyᵢₛ from the second difference value Δyᵢₕ and dividing that result by the first difference value Δyᵢₛ, and the square root of

Δyᵢₛ/y̅ᵢ

corresponding to the blood vessel volume.

The blood-pressure calculator 44 calculates Rᵢ corresponding to a vascular resistance of the subject based on Equations (14) and (15).

$$\begin{matrix}{R}_{i} = \frac{{\overline{y}}_{i} - Δ {y}_{is}}{{y}_{i}^{′}} & ( 14 ) \\ {y}_{i}^{′} := {max}_{{t}_{0} < t < {t}_{2}} \frac{y ( t + 1 / fs ) - y ( t )}{1 / fs} & ( 15 )\end{matrix}$$

Here, fₛ is a sampling frequency of the pulse yᵢ.

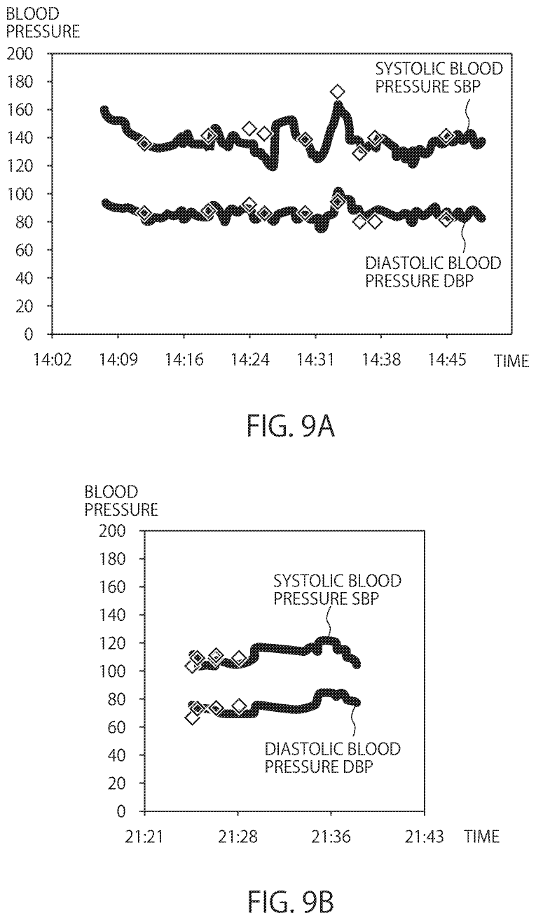

FIG. 9A is a diagram illustrating measured data of a subject having a relatively high blood pressure. FIG. 9B is a diagram illustrating measured data of a subject having a relatively low blood pressure. The vertical axis represents a blood pressure and the horizontal axis represents a time. Rhombic marks represent values of measurement by the blood-pressure measurement apparatus 1, and solid lines represent data measured by a medical instrument (a wrist-cuff type) for comparison. The values measured by the blood-pressure measurement apparatus 1 according to the present embodiment well coincide with data measured for comparison in both cases.

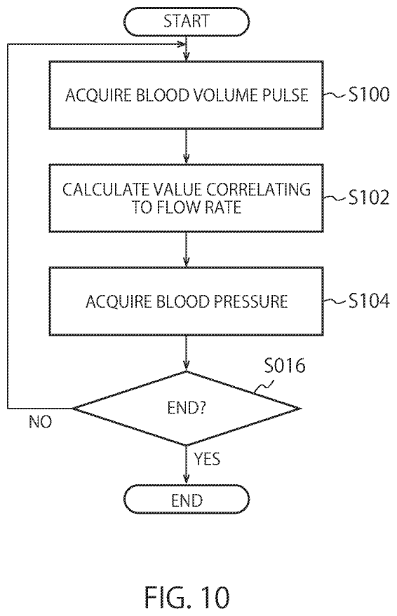

FIG. 10 is a flowchart illustrating processing by the blood-pressure measurement apparatus 1. First, the measurer 2 acquires a pulse of a subject (Step S100). Subsequently, the feature-point processor 42 performs processing based on the pulse yᵢ.

Next, the blood-pressure calculator 44 calculates the flow rate Q_{EL} corresponding to a blood flow of the subject from a time at which a value obtained by the first-order differentiation of the pulse yᵢ with respect to a time becomes the maximum to a time of the maximum peak of the pulse yᵢ, the resistance R corresponding to a vascular resistance of the subject, and the flow rate Q_{SE} corresponding to a blood flow from a rising time of the pulse to the time at which the value obtained by the first-order differentiation of the pulse with respect to the time becomes the maximum (Step S102).

Next, the blood-pressure calculator 44 calculates the diastolic blood pressure DBP, the systolic blood pressure SBP, and the pulse pressure PP based on the flow rate Q_{EL}, the resistance R, and the flow rate Q_{SE} (Step S104). The blood-pressure calculator 44 determines whether to end the overall processing (Step S106), ends the overall processing when the overall processing is determined to be ended (Step S106: YES), and repeats the processes from Step S100 when the overall processing is determined not to be ended (Step S106: NO).

As described above, the systolic blood pressure SBPᵢ is acquired based on Equations (5) and (10) and the diastolic blood pressure DBPᵢ is acquired based on Equations (6) and (13) in the present embodiment. Therefore, it is possible to simply and accurately detect a blood pressure.

### Second Embodiment

While the blood-pressure measurement apparatus 1 according to the first embodiment calculates the systolic blood pressure SBP based on the flow rate Q_{SE} (FIG. 6), the blood-pressure measurement apparatus 1 according to a second embodiment calculates the systolic blood pressure SBP also based on the flow rate Q_{EH}. Further, the blood-pressure measurement apparatus 1 according to the first embodiment is different from the blood-pressure measurement apparatus 1 according to the second embodiment in that, while the blood-pressure measurement apparatus 1 according to the first embodiment calculates the diastolic blood pressure DBP based on the flow rate Q_{EH} (FIG. 6), the blood-pressure measurement apparatus 1 according to the second embodiment calculates the diastolic blood pressure DBP based on a flow rate Q_{HD}. In the following descriptions, different points from the first embodiment are described.

A blood pressure measured by the blood-pressure measurement apparatus 1 according to the first embodiment well coincides with a systolic blood pressure SBP and a diastolic blood pressure DBP of ordinary people. However, it has been found that there are some subjects who have different pulse characteristics from ordinary people. The blood-pressure measurement apparatus 1 according to the second embodiment is configured to be able to treat such subjects.

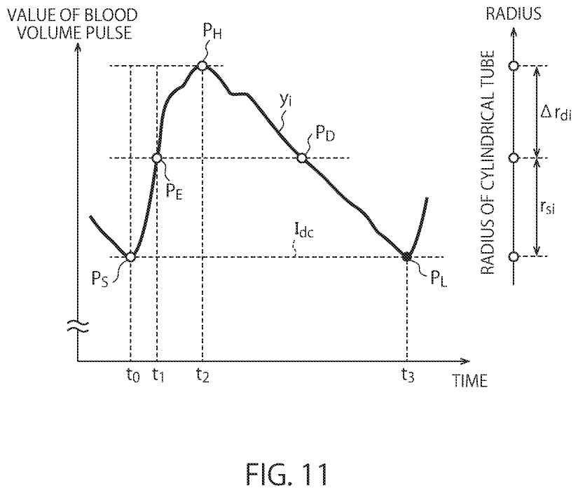

FIG. 11 is a diagram illustrating a relation between an example of a pulse waveform and a radius of a cylindrical tube model according to the second embodiment. The left part of FIG. 11 illustrates an example of a normal pulse waveform for one beat, similarly to FIG. 5. The vertical axis represents a value of pulse and the horizontal axis represents a time. The right part illustrates the radius of the cylindrical tube model. A change of a blood vessel volume is represented by a radius rₛᵢ and a change Δr_{di}. A point P_{D} is a point between the point P_{H} and the point P_{L}, which has the same value of a blood volume pulse as the point P_{E}. I_{dc} is a direct-current component of the blood volume pulse. Here, the suffix i is the number for identifying each pulse in blood volume pulse data. That is, the suffix i indicates data corresponding to a pulse at the i-th beat. A time of the point P₀ corresponds to a fifth reference time.

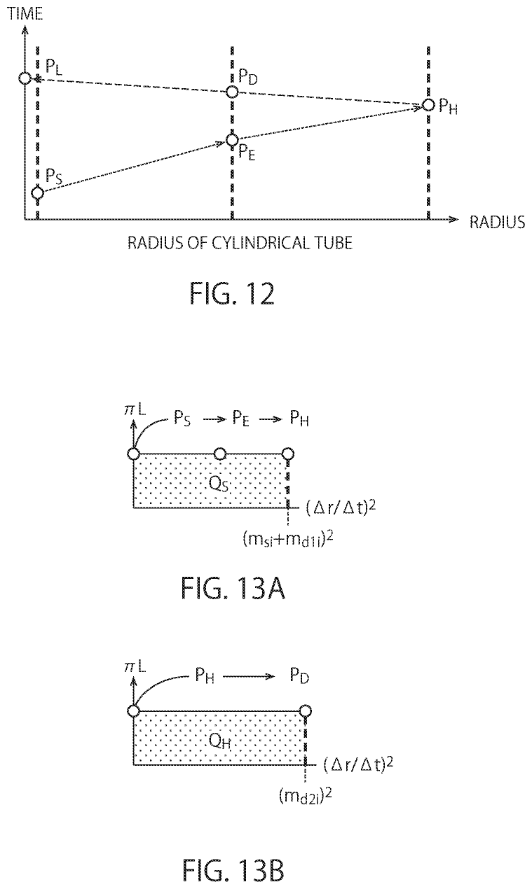

FIG. 12 is a diagram schematically illustrating a change of a radius of a cylindrical tube which is associated with a change of a blood vessel volume from the point P_{S} to the point P_{L}. That is, FIG. 12 illustrates the change of the radius of the cylindrical tube in association with a pulse for one beat. The vertical axis represents a time and the horizontal axis represents a change of the radius from the point P_{S}. The radius increases from the point P_{S} to the point P_{H} with the time, and thereafter decreases.

FIG. 13A is a diagram schematically illustrating a flow rate Q_{S} from the point P_{S} to the point P_{H} via the point P_{E}. FIG. 13B is a diagram schematically illustrating a flow rate Q_{HD} from the point P_{H} to a point P_{D}. The horizontal axis represents the square of an average rate of change of the radius r of a blood vessel (Δr/Δt), and the vertical axis represents a value obtained by multiplying the length L and n. mₛᵢ in FIG. 13A is an average rate of change of a radius from the point P_{S} to the point P_{E}, and m_{d1i} is an average rate of change of the radius from the point P_{E} to the point P_{H}. m_{d2i} in FIG. 13B is an average rate of change of the radius from the point P_{H} to the point P_{D}. The flow rate Q_{HD} according to the present embodiment corresponds to the first value, the resistance R corresponds to the second value, and the flow rate Q_{S} corresponds to the third value.

In the present embodiment, the systolic blood pressure SBP is calculated by using the flow rate Q_{S}. Expansion of a blood vessel diameter to the point P_{E} in a systolic phase of the heart is mainly caused by the Windkessel effect. After the point P_{E}, a restoring force and a damping force become dominant gradually. That is, in the present embodiment, a range of a force generated in the systolic phase of the heart is expanded up to the flow rate Q_{SE} from the point P_{E} at which the restoring force is added to the Windkessel effect to the point P_{H}, and the systolic blood pressure SBP is modeled. It is considered that there are some subjects for which the Windkessel effect appears more strongly also in the range from the point P_{E} to the point P_{H}. In a case of performing measurement also for such people, use of the flow rate Q_{S} can improve the measurement accuracy of the systolic blood pressure SBP. It is experimentally verified that, even if the flow rate Q_{S} is used, the accuracy of the systolic blood pressure SBP of ordinary people is not lowered.

Meanwhile, it is considered that, for the people for which the Windkessel effect appears more strongly in the range from the point P_{E} to the point P_{H}, a point at which the Windkessel effect becomes weak is shifted toward the point P_{L}. Since a diastolic blood pressure is a lower limit of a force by the Windkessel effect, the point at which the Windkessel effect becomes weak is shifted up to the point P_{H} and the diastolic blood pressure DBP is modeled by using a flow rate Q_{D} in the range from the point P_{H} to the point P_{D}. In particular, the flow rate Q_{D} is calculated based on the flow rate Q_{HD}. It is experimentally verified that, even if the flow rate Q_{HD} is used, the accuracy of the diastolic blood pressure DBP of ordinary people is not also lowered.

The model used by the blood-pressure acquirer 4 according to the present embodiment has been described above. An example of detailed processing by the blood-pressure acquirer 4 is described below.

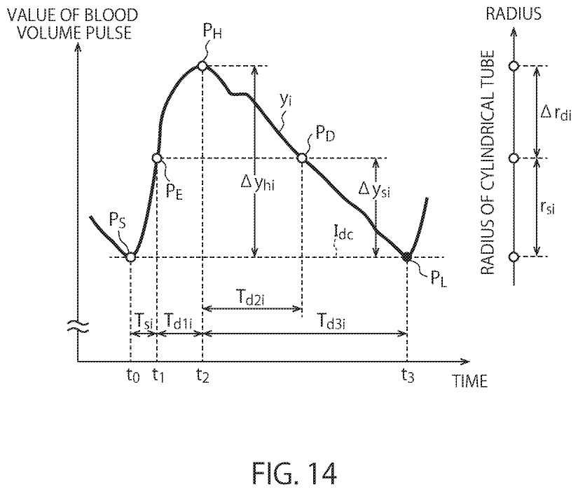

FIG. 14 is a diagram illustrating an example of information acquired from the pulse yᵢ by the feature-point processor 42 according to the second embodiment. The vertical axis represents a value of pulse and the horizontal axis represents a time. The right part of FIG. 14 illustrates a radius of a cylindrical tube model. A change of a blood vessel volume is represented by the radius rₛᵢ and the change Δr_{di}.

The feature-point processor 42 calculates the first difference value Δyₛᵢ obtained by subtracting the direct-current component I_{dc} from a value of the pulse yᵢ at the time t₁ and the second difference value Δyₕᵢ obtained by subtracting the direct-current component I_{dc} from a value of the pulse yᵢ at the time t₂.

Further, the feature-point processor 42 calculates a time T_{d2i} by using Equation (16). T_{d2i} is a time between the point P_{D} and the point PH. Tₛᵢ is a time obtained by subtracting to from the time t₁, T_{d1i} is a time obtained by subtracting t₁ from the time t₂, and T_{d3i} is a time obtained by subtracting t₂ from the time t₃. That is, the feature-point processor 42 acquires a time of the point P_{D} which has an equivalent value to the pulse yᵢ at the time t₁ in a time period from the time t₂ of the pulse yᵢ to the time t₃ as the fifth reference time, and calculates a time between the time t₂ and the fifth reference time as the time T_{d2i}.

$${T}_{d 2 i} \sim {T}_{d 3 i} \frac{Δ {y}_{hi} - Δ {y}_{si}}{Δ {y}_{hi}} \qquad ( 16 )$$

When a volume corresponding to the point P_{L} is assumed as a reference, Δyₛᵢ/I_{dc} is proportional to a volume at the points P_{E} and P_{D}, and similarly Δyₕᵢ/I_{dc} is proportional to a volume at the point P_{H}. G is a proportional constant, and I_{dc} is a value of a DC component of a pulse.

When the radius of the cylindrical tube changes from rₛᵢ to rₛᵢ+Δr_{di}, Δr_{di} can be calculated by Equations (17) to (19) by using the radius rₛᵢ at the point P_{E},

$$\begin{matrix}{V}_{i} = G Δ {y}_{ si} / {I}_{d c} & ( 17 ) \\ Δ {V}_{i} = G \frac{Δ {y}_{h i}}{{I}_{d c}} - {V}_{i} & ( 18 ) \\ Δ {r}_{di} = \frac{{T}_{r i}}{2} \frac{Δ {V}_{i}}{{V}_{i}} & ( 19 )\end{matrix}$$

Here, when the radius rₛᵢ in Equation (19) is arranged by using Equation (17), it can be deformed to Equations (20) and (21) described below. The blood-pressure calculator 44 calculates the radius rₛᵢ and Δr_{di} by using Equations (20) and (21). L is the length of the cylindrical tube model.

$$\begin{matrix}{r}_{si} = \sqrt{\frac{G}{L}} \sqrt{\frac{Δ {y}_{si}}{π {I}_{d c}}} & ( 20 ) \\ Δ {r}_{di} = \frac{1}{2} \frac{Δ {y}_{hi} - Δ {y}_{s i}}{Δ {y}_{si}} \sqrt{\frac{G}{L}} \sqrt{\frac{Δ {y}_{s i}}{π {I}_{di}}} & ( 21 )\end{matrix}$$

The blood-pressure calculator 44 calculates the average rate of change ms by using Equation (22).

$${m}_{si} = \frac{{r}_{si}}{{T}_{si}} \qquad ( 22 )$$

The blood-pressure calculator 44 also calculates the average rates of change m_{d1i} and m_{d2i} by using Equations (23) and (24), respectively.

$$\begin{matrix}{m}_{d 1 i} = \frac{Δ {r}_{di}}{{T}_{d 1 i}} & ( 23 ) \\ {m}_{d 2 i} = \frac{Δ {r}_{di}}{{T}_{d 2 i}} & ( 24 )\end{matrix}$$

The blood-pressure calculator 44 calculates a flow rate Qₛᵢ based on the average rates of change m_{d1i} and m_{d2i} by using Equation (25).

\sqrt{Qₛᵢ}=πL(mₛᵢ+m_{di})² (25)

The blood-pressure calculator 44 calculates the resistance Rᵢ by using Equation (26). Here, Vᵢ is a volume of the cylindrical tube model, and Vᵢ(t₁) is a volume of the cylindrical tube model at the time t₁. That is, I_{dc} corresponds to in the first embodiment. Accordingly, Equation (26) has an equivalent value to Equation (16).

$$\text{?} = \text{?} \\ \text{?} \text{indicates text missing or illegible when filed} \qquad ( 26 )$$

The blood-pressure calculator 44 calculates the flow rate Q_{Di} from the point P_{H} to the point P_{L} based on Equation (27) by using the resistance Rᵢ and the compliance C.

$$\begin{matrix}\sqrt{{Q}_{Di}} = {Q}_{HDi} {e}^{- {T}_{d 3 i} / {R}_{di} C} \\ = π {L ( {m}_{d 2 i} )}^{2} {e}^{- {T}_{d 3 i} / {R}_{di} C} \\ = G \frac{{( Δ {y}_{hi} - Δ {y}_{si} )}^{2}}{4 {I}_{d c} Δ {{y}_{si} ( {T}_{d 2 i} )}^{2}} {e}^{- {T}_{d 3 i} / {R}_{di} C}\end{matrix} \qquad ( 27 )$$

The blood-pressure calculator 44 calculates R_{di} corresponding to a vascular resistance of the subject based on Equations (28) and (29).

$$\begin{matrix}{y}_{di}^{′} = \frac{{y}_{i} ( {t}_{2} + {T}_{d 2 i} + i / f s ) - {y}_{i} ( {t}_{2} + {T}_{d 2 i} )}{1 / fs} & ( 28 ) \\ {R}_{d i} = \frac{\vert {y}_{di}^{′} \vert}{{y}_{i}^{′}} & ( 29 )\end{matrix}$$

The blood-pressure calculator 44 calculates the diastolic blood pressure DBP and the systolic blood pressure SBP at each i-th heartbeat based on Equations (30) and (31).

ln DBPᵢ=a₁ ln Q_{Di}+a₂ ln R_{di}+α (30)

ln SBPᵢ=b₁ ln Q_{Si}+b₂ ln R_{di}+β+ln DBPᵢ (31)

Here, a₁, a₂, b₁, b₂ α, and β are constants.

As described above, the blood-pressure calculator 44 acquires the diastolic blood pressure DBP based on the flow rate Q_{HD} (the first value) corresponding to a blood flow of a subject in the time period T_{d2i} (a first time period) in a time period from the first time t₁ at which a value obtained by the first-order differentiation of the pulse yᵢ with respect to a time becomes the maximum to a fourth time t₃ at which a next pulse rises, and Rdᵢ (the second value) corresponding to a vascular resistance of the subject. Further, the blood-pressure calculator 44 acquires the systolic blood pressure further based on the flow rate Q_{S} (the third value) corresponding to a blood flow of the subject in a time period (Tₛᵢ+T_{d1i}) (a second time period) in a time period from the third time t₀ at which the pulse rises to the second time t₂ of the maximum peak of the pulse. The first time according to the present embodiment corresponds to the first reference time, the second time corresponds to the fourth reference time, the third time corresponds to the third reference time, and the fourth time corresponds to a second reference time.

As described above, the diastolic blood pressure DBPᵢ is acquired based on Equations (27) and (30) and the systolic blood pressure SBPᵢ is acquired based on Equations (25) and (31) in the present embodiment. Therefore, it is possible to simply and accurately detect a blood pressure.

At least a part of the blood-pressure measurement apparatus 1 may be constituted by hardware or software. When the apparatus is constituted by software, it is possible to configure that a program for realizing at least a part of the functions of the blood-pressure measurement apparatus 1 is held in a recording medium such as a flexible disk or a CD-ROM and a computer is caused to read and execute the program. The recording medium is not limited to a detachable one such as a magnetic disk or an optical disk, and a stationary recording medium such as a hard disk device or a memory may be also applicable.

Further, the program for realizing at least a part of the functions of the blood-pressure measurement apparatus 1 may be distributed via a communication line (including wireless communication) such as the Internet. Furthermore, the program may be distributed via a wired line or a wireless line such as the Internet or distributed while being held in a recording medium, in a state where the program is encrypted, modulated, or compressed.

While certain embodiments have been described, these embodiments have been presented by way of example only, and are not intended to limit the scope of the inventions. Indeed, the novel methods and systems described herein may be embodied in a variety of other forms; furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

1. A blood-pressure measurement apparatus comprising:
a measurer configured to measure a pulse of a subject based on a received-light signal scattered in a body of the subject when a light signal in a predetermined frequency band is irradiated; and
a blood-pressure acquirer configured to acquire a diastolic blood pressure based on a first value and a second value, the first value corresponding to a blood flow of the subject in a first time period in a time period from a first reference time at which a value obtained by first-order differentiation of the pulse with respect to a time becomes a maximum to a second reference time at which a next pulse rises, the second value corresponding to a vascular resistance of the subject.

2. The apparatus of claim 1, wherein the blood-pressure acquirer acquires a systolic blood pressure further based on a third value corresponding to a blood flow of the subject in a second time period in a time period from a third reference time at which the pulse rises to a fourth reference time of a maximum peak of the pulse.

3. The apparatus of claim 2, wherein the first time period is between the first reference time and the fourth reference time, and the second time period is a time period from the third reference time to the first reference time.

4. The apparatus of claim 2, wherein the blood-pressure acquirer obtains a fifth reference time that has an equivalent value to the pulse at the first reference time in a time period from the fourth reference time to the second reference time, and
the first time period and the second time period are a time period from the fourth reference time to the fifth reference time.

5. The apparatus of claim 2, wherein the second time period is a time period from the third reference time to the first reference time.

6. The apparatus of claim 4, wherein the second time period is a time period from the fourth reference time to the fifth reference time.

7. The apparatus of claim 2, wherein blood-pressure acquirer acquires the third reference time based on a value obtained by dividing a first difference value obtained by subtracting a direct-current component of the pulse from a value of the pulse at the first reference time, by the maximum value of the first-order differentiation.

8. The apparatus of claim 2, wherein the blood-pressure acquirer acquires at least one of the first value and the third value based on a value corresponding to a blood vessel volume of the subject at the first reference time.

9. A blood-pressure measurement method comprising:
measuring a pulse of a subject based on a received-light signal that is scattered in a body of the subject and is then received when a light signal in a predetermined frequency band is irradiated to the subject; and
acquiring a diastolic blood pressure based on a first value and a second value, the first value corresponding to a blood flow of the subject in a first time period in a time period from a first reference time at which a value obtained by first-order differentiation of the pulse with respect to a time becomes a maximum to a second reference time at which a next pulse rises, the second value corresponding to a vascular resistance of the subject.

10. The method of claim 9, wherein the blood-pressure acquiring acquires a systolic blood pressure further based on a third value corresponding to a blood flow of the subject in a second time period in a time period from a third reference time at which the pulse rises to a fourth reference time of a maximum peak of the pulse.

11. The method of claim 10, wherein the first time period is between the first reference time and the fourth reference time, and the second time period is a time period from the third reference time to the first reference time.

12. The method of claim 10, wherein the blood-pressure acquiring obtains a fifth reference time that has an equivalent value to the pulse at the first reference time in a time period from the fourth reference time to the second reference time, and
the first time period and the second time period are a time period from the fourth reference time to the fifth reference time.

13. The method of claim 10, wherein the second time period is a time period from the third reference time to the first reference time.

14. The method of claim 12, wherein the second time period is a time period from the fourth reference time to the fifth reference time.

15. The method of claim 10, wherein blood-pressure acquiring acquires the third reference time based on a value obtained by dividing a first difference value obtained by subtracting a direct-current component of the pulse from a value of the pulse at the first reference time, by the maximum value of the first-order differentiation.

16. The method of claim 10, wherein the blood-pressure acquiring acquires at least one of the first value and the third value based on a value corresponding to a blood vessel volume of the subject at the first reference time.
